(19) United States
(12) Patent Application Publication
(10) Pub. No.: US 2026/0178099 A1
(43) Pub. Date: Jun. 25, 2026

(54) IN-USE EQUIPMENT ESTIMATION DEVICE

(71) Applicant: NTT DOCOMO, INC., Tokyo (JP)

(72) Inventors: Masayasu SUMIYA, Chiyoda-ku (JP); Fumiaki IGARASHI, Chiyoda-ku (JP)

(73) Assignee: NTT DOCOMO, INC., Tokyo (JP)

(21) Appl. No.: 19/112,691

(22) PCT Filed: Oct. 10, 2023
(86) PCT No.: PCT/JP2023/036781

§ 371 Date: Mar. 18, 2025

(30) Foreign Application Priority Data
Oct. 26, 2022 (JP) 2022-171267

(51) Int. Cl. G06F 1/3203; A61B 5/16; G06F 1/3206

(52) U.S. Cl. CPC G06F 1/3203; A61B 5/165; G06F 1/3206

(57) ABSTRACT

Provided is an in-use equipment device for easily estimating the use of in-use equipment such as home appliances without using sensors that only collect power consumption. In a management device 100, a communication unit 101 acquires a user's biometric information from a wearable device 120 worn by the user. The acquired biometric information is stored in a log database 104. When an estimation timing is reached, an estimation unit 102 estimates the equipment being used by the user, on the basis of the biometric information stored in the log database 104.

### TECHNICAL FIELD

The present invention relates to an in-use equipment estimation device for estimating a use state of equipment such as a home appliance.

### BACKGROUND ART

Interest in SDGs has increased globally, and in Japan, in particular, there has been a growing movement to reduce energy consumption due to the country's low energy self-sufficiency rate. Moreover, in recent years, home power consumption has increased because of the increase in remote work, voluntary refraining from going out, and the like due to the effects of the COVID-19 pandemic, and home power saving to suppress energy consumption at home has attracted attention.

Although a device that visualizes home power consumption has been provided as an existing solution to promote home power saving, examples of the drawbacks of this device are as follows. For example, displaying the overall aggregate value of electricity used at home is effective for ascertaining the overall picture. However, it is not possible to ascertain the trend in the power consumption of each home appliance and it is difficult to automatically propose effective power saving methods and the like. Moreover, the overall aggregate value of electricity used at home can be collected by collecting information from a smart meter. However, in order to ascertain the power consumption of individual home appliances, it is necessary to install a sensor capable of communication at the outlet of each home appliance, and the installation of a number of sensors solely for power collection without other applications increases the financial and operational burden on users, which may be a factor that reduces their motivation to save energy and the like.

Patent Literature 1 describes technology for predicting an amount of used power and searching for and displaying a proposal for changing a usage time period of at least one home appliance estimated to be in use when the predicted amount of used power exceeds a reference amount.

Moreover, in Patent Literature 2, a home appliance state estimation device that does not require special work such as installation work and is capable of estimating the state of general home appliances other than high-function appliances is disclosed.

### CITATION LIST

### Patent Literature

[Patent Literature 1] Japanese Unexamined Patent Publication No. 2005-253340

### SUMMARY OF INVENTION

### Technical Problem

The technology described in Patent Literature 1 is based on the premise that information about a change in power data when the consumer's electrical appliances are operated is acquired in advance to estimate the home appliances in use. Thereby, because it is necessary to require that the power consumption trends of each home appliance owned by the consumer be measured in advance, the installation of a measuring device or the like is required and there is a problem that the introduction cost may be increased.

Moreover, in the technology described in Patent Literature 2, the use of sensors provided in PCs, tablets, smartphones, and the like is disclosed.

However, because these PCs and the like are not always turned on or are not necessarily placed near each appliance, these sensors cannot determine the external state of each appliance using a temperature sensor, a microphone, or the like all the time.

Therefore, in order to solve the above-mentioned problems, an objective of the present invention is to provide an in-use equipment estimation device for easily estimating the use of in-use equipment such as home appliances without using sensors that only collect power consumption.

### Solution to Problem

According to the present invention, there is provided an in-use equipment estimation device including: a biometric information acquisition unit configured to acquire a user's biometric information from a wearable device worn by the user; and an estimation unit configured to estimate equipment being used by the user on the basis of the biometric information.

### Advantageous Effects of Invention

According to the present invention, it is possible to easily estimate the use of in-use equipment such as home appliances.

### BRIEF DESCRIPTION OF DRAWINGS

FIG. 1 is a diagram showing a system configuration in a home including a management device 100 which is an in-use equipment estimation device according to the present disclosure.

FIG. 2 is a block diagram showing a functional configuration of the management device 100.

FIG. 3 is a diagram showing a specific example of a log database 104.

FIG. 4 is a schematic diagram of an estimation process of an estimation model 103.

FIG. 5 is a flowchart showing an operation of the management device 100.

FIG. 6 is a diagram showing an example of display on a display unit 105.

FIG. 7 is a diagram showing fixed power consumption or changed power consumption for each time period.

FIG. 8 is a flowchart (part 1) showing a process based on classification rules for fixed power consumption and changed power consumption.

FIG. 9 is a flowchart (part 2) showing the process based on the classification rules for the fixed power consumption and the changed power consumption.

FIG. 10 is a diagram showing a system configuration including a management device 100a according to a modified example.

FIG. 11 is a diagram showing an example of display on a log database 104 and a display unit 105 when an in-use equipment entry is discarded.

FIG. 12 is a diagram showing an example of a hardware configuration of a management device 100 according to an embodiment of the present disclosure.

### DESCRIPTION OF EMBODIMENTS

Embodiments of the present disclosure will be described with reference to the accompanying drawings. If possible, the same parts are denoted by the same reference signs and redundant description thereof will be omitted.

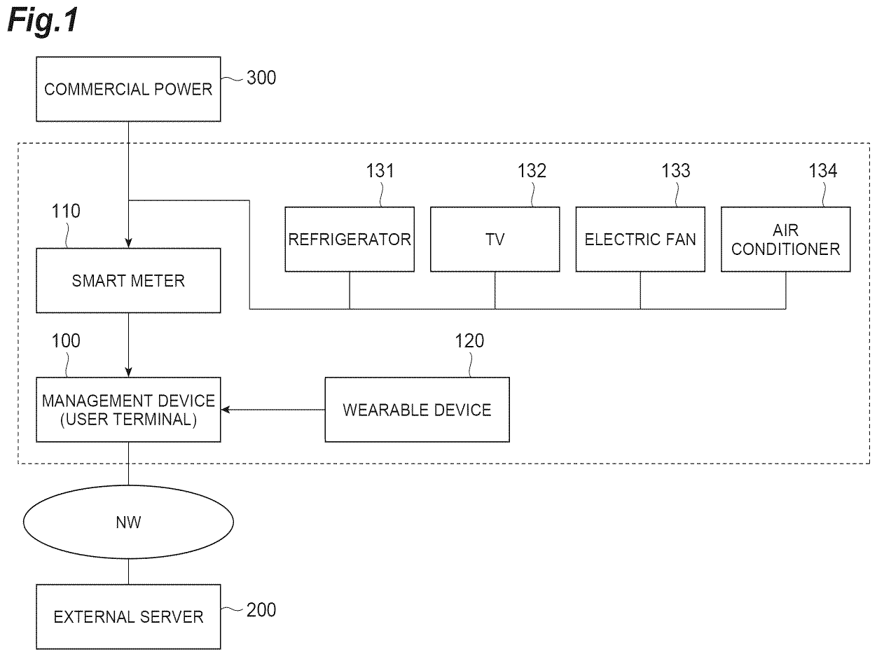

FIG. 1 is a diagram showing a system configuration in a home including a management device 100, which is an in-use equipment estimation device of the present disclosure. As shown in FIG. 1, this system includes a management device 100, a wearable device 120, and an external server 200.

In the home, there is in-use equipment such as a refrigerator 131, a TV 132, an electric fan 133, an air conditioner 134, and commercial power 300 supplies power to the in-use equipment. A smart meter 110 measures power consumption of the in-use equipment.

The wearable device 120 is a device that acquires biometric information of the user (a body temperature, pulse, blood pressure, and the like).

The management device 100 acquires power consumption information of each in-use equipment item from the smart meter 110 and acquires biometric information or the user's behavior (the number of steps, a body tilt, whether the user is active, and the like) from the wearable device 120. Also, the management device 100 determines whether or not the in-use equipment such as the refrigerator 131 is in use on the basis of the power consumption information and biometric information.

Moreover, the management device 100 obtains weather information (weather, temperature, humidity, and the like) from the external server 200 and estimates an operating situation of the in-use equipment on the basis of the weather information (external information) along with the biometric information, power consumption, and the like.

The external server 200 is a server that stores weather information (weather-related information such as weather, temperature, humidity, and precipitation). This external server 200 is a server that stores weather information that is generally available.

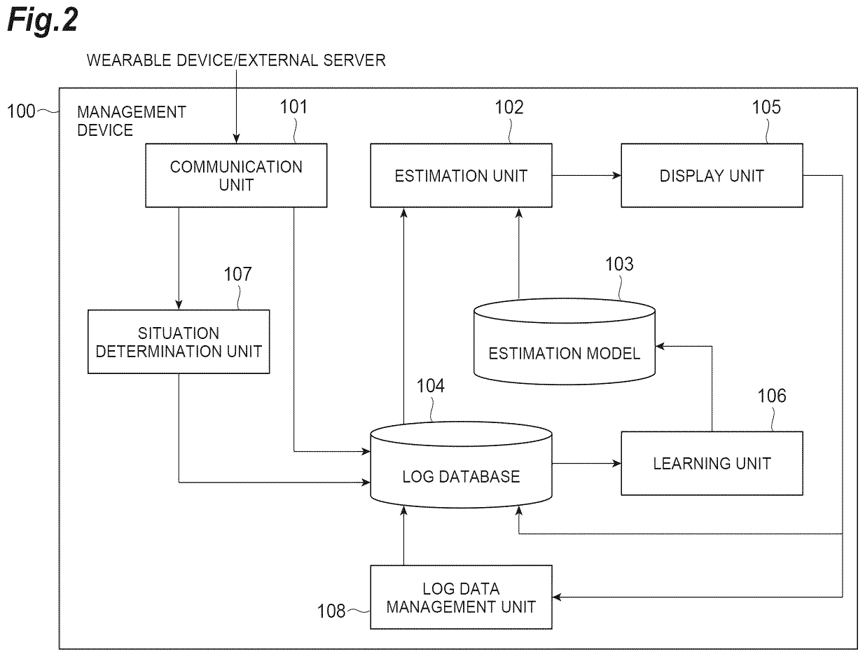

FIG. 2 is a block diagram showing a functional configuration of the management device 100. As shown in FIG. 2, the management device 100 is configured to include a communication unit 101, an estimation unit 102, an estimation model 103, a log database 104, a display unit 105, a learning unit 106, a situation determination unit 107, and a log data management unit 108.

The communication unit 101 is a part that acquires biometric information and external information (weather information, the user's behavior, power consumption information, and the like) from the wearable device 120 and the external server 200.

The estimation unit 102 is a part that estimates a use situation of in-use equipment using the estimation model 103 and the log database 104. That is, the estimation unit 102 inputs the user's most recent biometric information and the most recent external information stored in the log database 104 to the estimation model 103, and acquires the use situation of the in-use equipment (whether or not the equipment is in use) output from the estimation model 103. The external information may be weather information, the user's behavior, power consumption, and a power consumption type, but all of these are not necessarily required, and only at least one of them is necessary.

The estimation unit 102 may input information such as biometric information to the estimation model 103 as it is or may derive a change state (an increase, a decrease, or the like) from the most recent state and input the change state to the estimation model 103.

The estimation model 103 is a machine learning model that inputs the biometric information of the user and the external information stored in the log database 104 and outputs the use situation of the in-use equipment. This estimation model 103 is stored in a storage unit (not shown). For convenience, the biometric information, the external information, and the like are stored in the log database 104, but the estimation unit 102 may directly acquire them from the communication unit 101 and input them to the estimation model 103.

The log database 104 is a part that stores the biometric information acquired by the communication unit 101, the weather information acquired from the external server 200, and other external information. Moreover, the log database 104 stores results of a survey conducted in advance in association with a date and time and the like. These survey results may come from a single user serving as an estimation target or may include results from other users.

The display unit 105 is a part that displays the use situation of the in-use equipment and asks the user about the success or failure of the use situation. For example, the display unit 105 includes a touch panel display, asks the user about the success or failure of the use situation of the in-use equipment, and receives a response to the inquiry. This response is reflected and stored in the log database 104.

The learning unit 106 is a part that trains and updates the estimation model 103. The learning unit 106 generates the estimation model 103 by designating the log data stored in the log database 104 as an explanatory variable, designating the in-use equipment corresponding to the above-described survey result or response as an objective variable, and performing a learning process using the known machine learning.

The situation determination unit 107 is a part that determines whether the power consumption is fixed or changed on the basis of the user's behavior and the like. That is, the situation determination unit 107 determines the power consumption type on the basis of the location information, residence, activity state, power consumption, and the like of the user (management device 100). Information about this power consumption type is used in the estimation process.

Next, an operation of the estimation unit 102 will be described. As described above, the estimation unit 102 estimates the use situation of the in-use equipment in the user's home on the basis of the user's biometric information, weather information, and power consumption information.

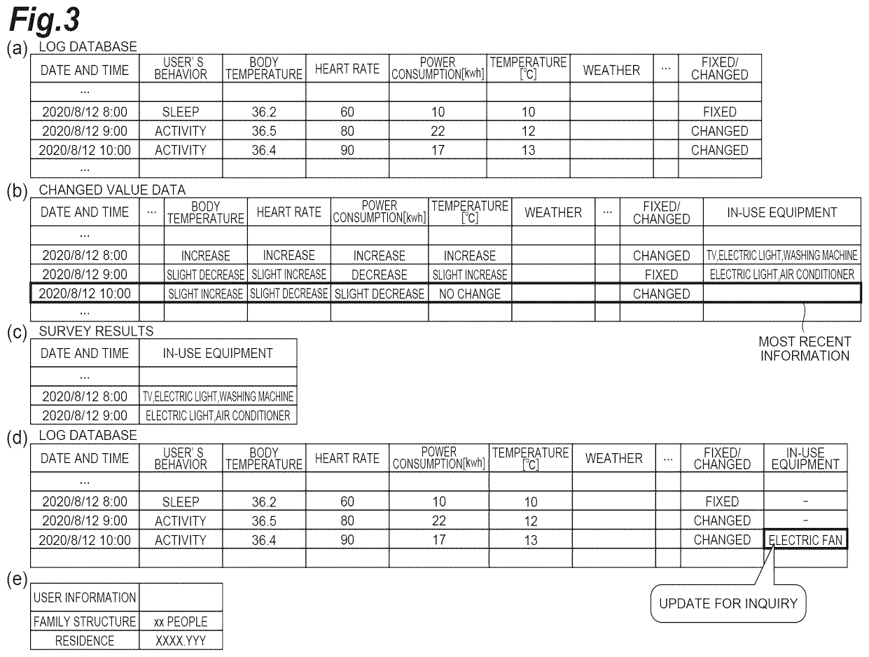

The estimation unit 102 processes the data in the log database 104 before the estimation process. FIG. 3 is a diagram showing a specific example. FIG. 3(a) shows a specific example of log data of a target user stored in the log database 104. As shown in FIG. 3, the log database 104 stores a date and time of measurement, the user's behavior (activity and sleep) at that time, the user's biometric information (the user's body temperature and the user's heart rate and blood pressure), the power consumption obtained from the smart meter 110, the temperature, the weather, and the power consumption type (fixed/changed) acquired from the external server 200, and the like.

The estimation unit 102 processes log data into changed value data indicating a change state (an increase, a decrease, maintenance, or the like). FIG. 3(b) is a diagram showing the body temperature, heart rate, power consumption, temperature, and the like processed into information indicating the change state such as the increase or the decrease for each hour. The increase or decrease in power consumption further indicates whether it is an increase or a decrease in fixed power consumption or an increase or a decrease in changed power consumption. The estimation unit 102 inputs the changed value data to the estimation model 103 and receives the output of the use situation of the in-use equipment from the estimation model 103. The changed power consumption and the fixed power consumption will be described below.

The changed value data shown in FIG. 3(b) is designated as an explanatory variable, the in-use equipment at that time is designated as an objective variable, and a learning process is performed to generate the estimation model 103. At this time, the in-use equipment is prepared in advance by a survey or the like. For example, the body temperature, heart rate, fixed power consumption, and air temperature increase between 8:00 and 9:00 on Aug. 12, 2020 in FIG. 3(b). At this time, it is assumed that information indicating that the TV has been turned on is obtained from the survey result (see FIG. 3(c)).

FIG. 3(c) shows the survey result. The in-use equipment of the user is registered in advance here. The in-use equipment used here is reflected in an in-use equipment field in FIG. 3(b) and is used as learning data.

The learning unit 106 performs machine learning to train the estimation model 103 using a situation in which the body temperature, the heart rate, the fixed power consumption, or the air temperature increases as an explanatory variable and a situation in which the TV is turned on as an objective variable. The objective variable is a situation in which the TV is turned on in the above description, but is not limited thereto. The objective variable may also be the use situation of a plurality of equipment items, such as a situation in which the TV is turned on and a situation in which electricity is turned on.

In this way, the learning unit 106 can perform machine learning on the basis of changed value data and a survey result (in-use equipment) to generate the estimation model 103.

In addition, when the estimation model 103 is initially generated, the learning unit 106 uses a survey result to acquire in-use equipment serving as a learning target, but after the estimation model 103 is generated, the log database 104 is updated in accordance with presentation of the in-use equipment estimated for the user and its response, and the learning process is performed on the basis of the updated log database 104.

FIG. 3(d) is a diagram showing a specific example of the updated log database 104. As shown in FIG. 3(d), no survey results have been obtained for a time period after 10:00 on Aug. 12, 2020, but the in-use equipment can be obtained by estimating the in-use equipment using the estimation model 103 trained up to that time and asking the user about the result. Subsequently, the learning unit 106 periodically performs a learning process again on the basis of the log database 104 or results of a survey that has been performed again and updates the estimation model 103.

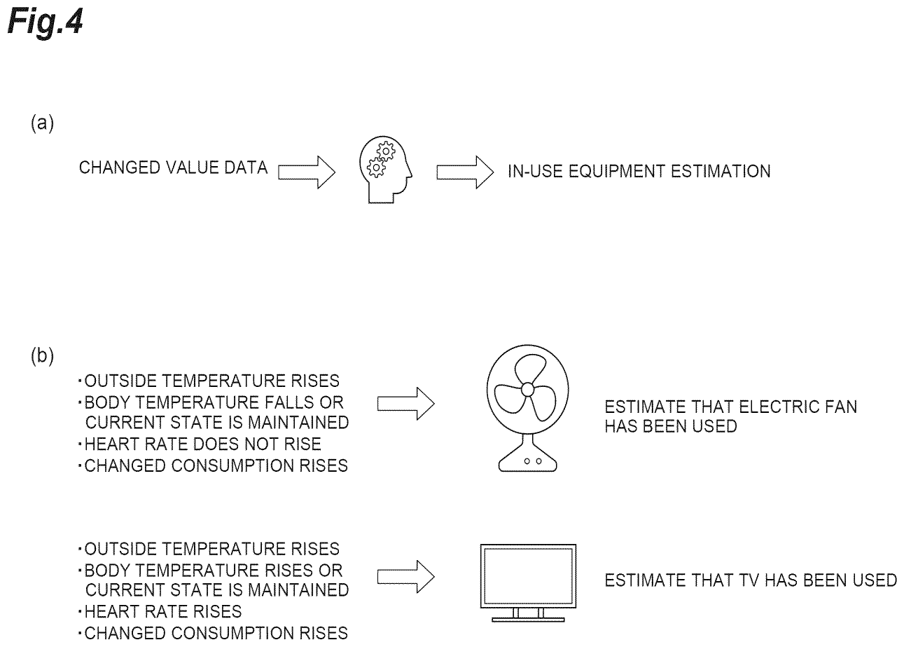

FIG. 4(a) is a schematic diagram of the estimation process of the estimation model 103. The estimation model 103 is trained on the basis of the changed value data and the survey results/response (in-use equipment). For example, as shown in FIG. 4(b), if the outside temperature rises, the body temperature falls (or the current state is maintained), the heart rate does not rise, and the changed power consumption rises, the estimation model 103 estimates that an electric fan has been used. Likewise, when the outside temperature rises, the body temperature rises (or the current state is maintained), the heart rate rises, and the changed power consumption rises, the estimation model 103 estimates that a TV has been used.

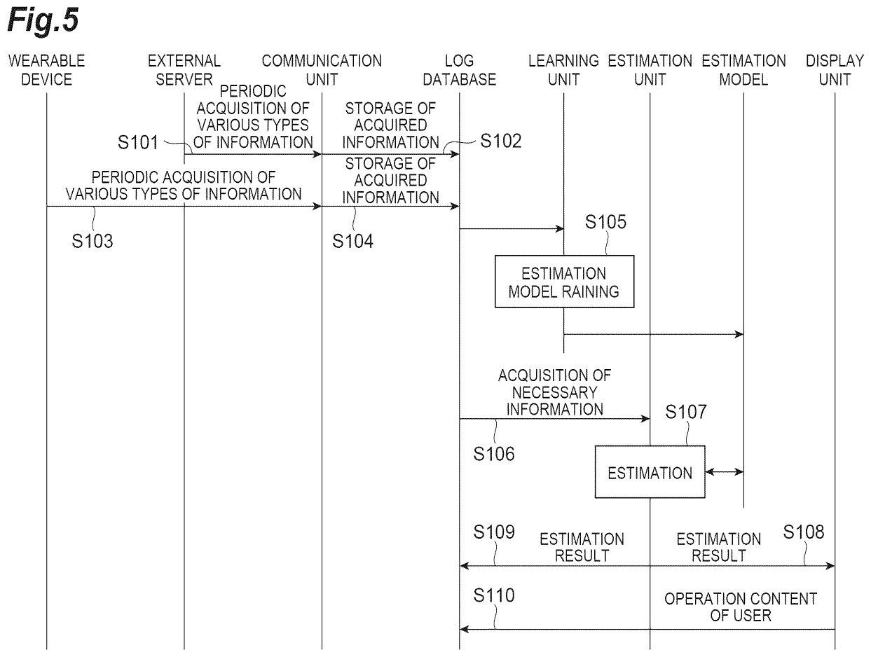

Next, the operation of the management device 100 in the present disclosure will be described. FIG. 5 is a flowchart showing an operation of the management device 100. The communication unit 101 periodically acquires various types of information (for example, weather information such as external information) from the external server 200 (S101). Moreover, the communication unit 101 also periodically acquires various types of information (biometric information) from the wearable device 120 (S103). Furthermore, when the initial estimation model 103 is generated, the communication unit 101 acquires survey result information from a survey information DB (not shown).

The communication unit 101 stores the acquired external information (weather information or the like) and biometric information in the log database 104 (S102 and S104). Initially, the survey result information is stored in the log database 104.

The learning unit 106 generates the estimation model 103 by performing a learning process (S105) and stores the estimation model 103 in the storage unit (not shown).

The processing of S101 to S105 is performed periodically in accordance with a predetermined timing and/or data update (when a certain number of records (in-use equipment) are obtained).

The estimation unit 102 acquires the most recent (for example, the past hour) necessary information (biometric information, weather information, and the like) from the log database 104 at a pre-designated timing (for example, every hour) (S106) and estimates the in-use equipment using the estimation model 103 (S107). The estimation unit 102 causes the display unit 105 to display the in-use equipment corresponding to an estimation result and requests the user to provide a response of whether or not the in-use equipment is correct (S108). Moreover, the estimation unit 102 outputs the estimation result to the log database 104 and registers the estimation result in the in-use equipment field for its time period (S109).

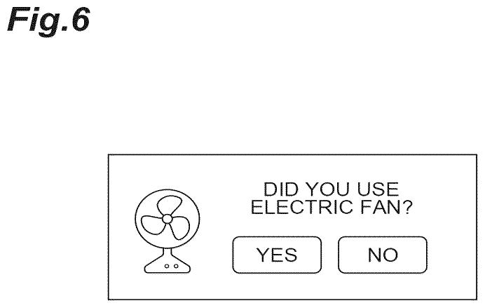

The user looks at the in-use equipment displayed on the display unit 105 and provides a response corresponding to an actual situation according to an operation of the user (S110). FIG. 6 shows an example of the display on the display unit 105. In FIG. 6, the user is asked whether or not the electric fan has been used. When the user is actually using the electric fan, YES is selected (tapped). Otherwise, ON is selected (tapped). Thereby, the actually used in-use equipment is reflected in the log database 104 from the display unit 105 and is used to update the estimation model 103.

The display unit 105 displays the in-use equipment of the estimation result so that the user can confirm the displayed in-use equipment at any timing. At this time, the display unit 105 may randomly select and display one of a plurality of in-use equipment items as an estimation result. The user can provide a response of whether or not the displayed in-use equipment has been used by operating the display unit 105.

The user's response result is stored in the log database 104 (see FIG. 3(d)). The learning unit 106 sequentially updates (retrains) the estimation model 103 when a certain number of responses (arbitrarily set, 30 or more records for both fixed power consumption and changed power consumption, and the like) are accumulated in the log database 104. At this time, it is possible to reflect general information while reflecting the user's tendency more by clearly distinguishing the user's response data from data other than the response data (a group of data obtained from subjects with several patterns).

For example, the initial estimation model 103 based on the survey results may not be a model specialized for the user because it is for general use. Therefore, when the estimation model 103 is updated, it is preferable to update the estimation model 103 specialized for the target user by reducing or eliminating the application of the survey results. For example, at the time of learning, the learning unit 106 performs a weighting process or the like, and retrieves only a response result from the target user from the log database 104 and performs a learning process.

There is a case where an existing entry of the in-use equipment at the time when the estimation model 103 was initially generated has been discarded. In contrast, there is also a case where newly added in-use equipment is present. In this case, the log data management unit 108 updates the in-use equipment stored in the log database 104. For example, when the user has added an entry of the in-use equipment, the log data management unit 108 stores the in-use equipment in the log database 104.

In contrast, when the user has discarded the entry of the in-use equipment, the log data management unit 108 deletes the in-use equipment from the log database 104.

Moreover, when the display unit 105 displays the discarded in-use equipment entry and the displayed discarded in-use equipment entry is indicated, the log data management unit 108 does not update the in-use equipment in the log database 104.

Next, fixed/changed power consumption will be described. The fixed power consumption indicates a power consumption type that does not change with the user's intention and the changed power consumption indicates a power consumption type that is likely to change with the user's intention. These power consumption types are classified based on rules. The fixed power consumption is power consumption mainly by a refrigerator. The changed power consumption is power consumption when the user's operation on a TV or the like is changed.

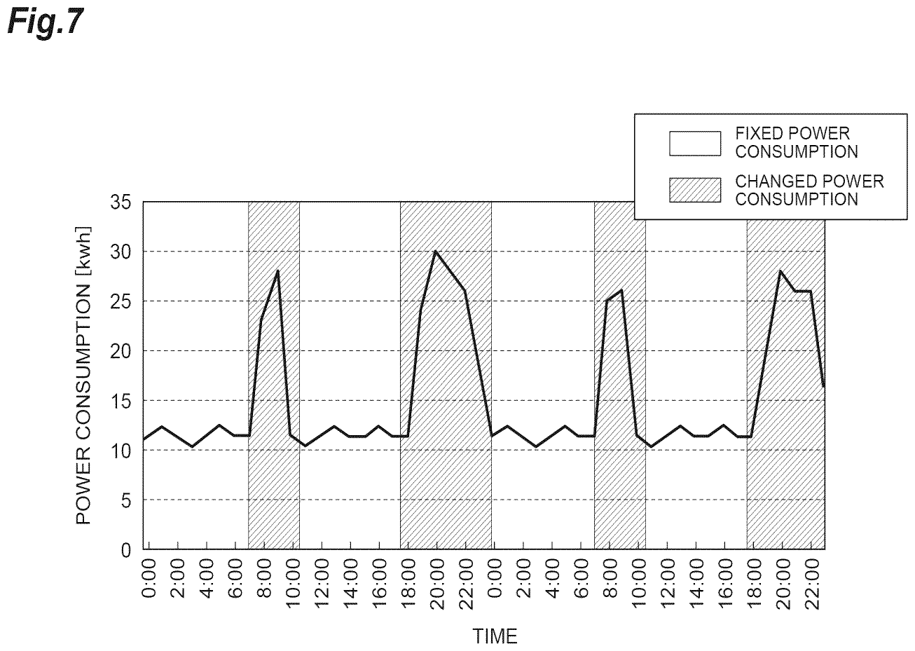

FIG. 7 is a diagram showing the fixed power consumption or the changed power consumption for each time period. As shown in FIG. 7, power consumption is classified as fixed power consumption or changed power consumption according to the time period.

In addition, information about a home appliance, a family structure, and the like used in the classification of power consumption types is based on a previous survey. Moreover, a user activity state for use in the classification is acquired from the wearable device 120.

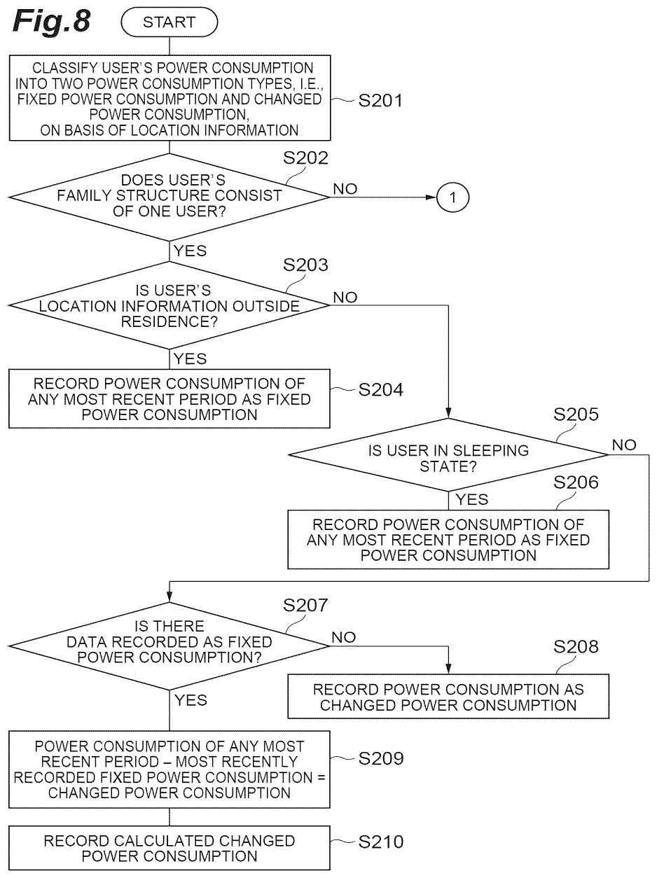

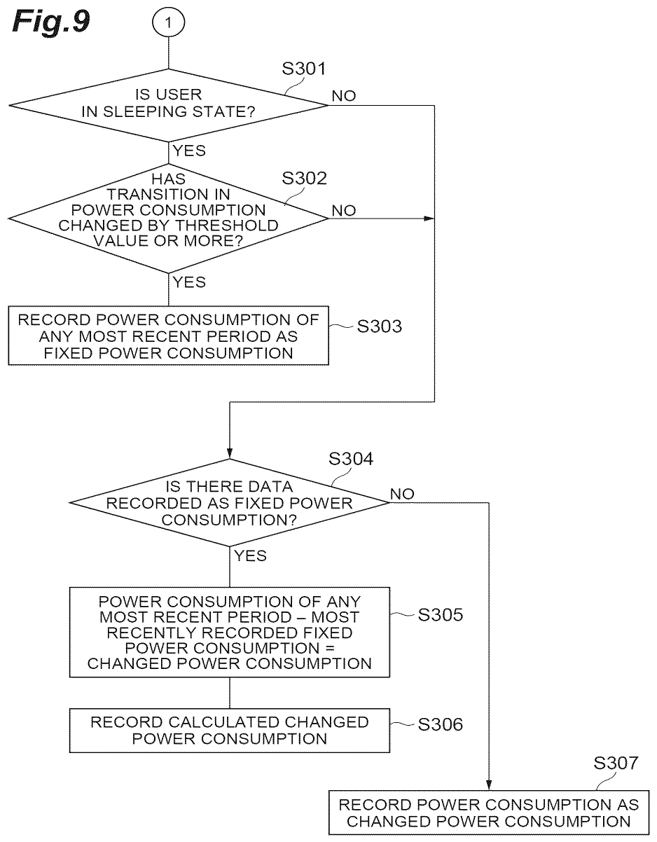

Next, the classification rules for fixed power consumption and changed power consumption will be described. FIGS. 8 and 9 are flowcharts showing a process based on the classification rules for the fixed power consumption and the changed power consumption. When a predetermined condition is satisfied (every hour in the present disclosure), the situation determination unit 107 starts a process of classifying the user's power consumption into two types, i.e., the fixed power consumption and the changed power consumption, based on the user's location information (S201). The situation determination unit 107 determines whether or not the user's family structure consists of one user with reference to the user information (FIG. 3(e)) in the log database 104 (S202). When it is determined that the family structure consists of one user (S202: YES), the situation determination unit 107 determines whether the user's location information is outside the residence (with reference to the user information) (S203).

When the situation determination unit 107 determines that the user's location information is outside the residence (S203: YES), the power consumption for any most recent period is recorded as fixed power consumption in the log database 104 (S204). For example, referring to FIG. 3(a) or 3(b), when the state at 9:00 on Aug. 12, 2020, is determined, power consumption of any most recent period (one hour between 8:00 and 9:00) is determined to be fixed power consumption. Subsequently, power consumption determination is made with reference to the log database 104.

When the situation determination unit 107 determines that the user's location information is in the residence (S203: YES), it is determined whether the user is in a sleeping state (S205). This is determined on the basis of a gyro sensor of the wearable device 120 and the like. When the situation determination unit 107 determines that the user is in a sleeping state (S205: YES), the power consumption of any most recent period is recorded as the fixed power consumption (S206).

The situation determination unit 107 determines whether or not there is data recorded as the fixed power consumption in the log database 104 (S207). When the situation determination unit 107 determines that there is no data recorded as fixed power consumption (S207: NO), the power consumption is recorded as the changed power consumption in the log database 104 (S208).

If there is data recorded as fixed power consumption (S207: YES), the situation determination unit 107 records it as (Power consumption of any most recent period−Most recently recorded fixed power consumption=Changed power consumption) (S209 and S210).

FIG. 9 is a flowchart showing a process of a case where the user's family structure does not consist of one person. If the situation determination unit 107 determines that the user is in a sleeping state (S301: YES), it is further determined whether the transition in power consumption has changed by a threshold value or more (S302). If the situation determination unit 107 determines that the transition in power consumption has not changed by the threshold value or more, the power consumption of any most recent period 104 is recorded in the log database 104 as the fixed power consumption (S303).

Moreover, if it is determined that the user is not in the sleeping state in step S301 or that the transition in power consumption has not changed by the threshold value or more in step S302, the situation determination unit 107 further determines whether or not there is data recorded in the log database 104 as fixed power consumption (S304).

Here, if it is determined that there is data recorded as the fixed power consumption (S304: YES), the situation determination unit 107 records it as (Power consumption of any most recent period−Most recently recorded fixed power consumption=Changed power consumption) (S305 and S306).

If the situation determination unit 107 determines that there is no data recorded as the fixed power consumption (S304: NO), the power consumption is recorded as the changed power consumption in the log database 104 (S307).

In this way, the situation determination unit 107 records the type in a fixed/changed field indicating the power consumption type in the log database.

Next, a modified example of the management device 100 of the present disclosure will be described. The management device 100 in FIG. 1 has been described as equivalent to a so-called user terminal (a smartphone or the like). The management device 100 is not limited to a user terminal and may be a device that functions as a server located on a network.

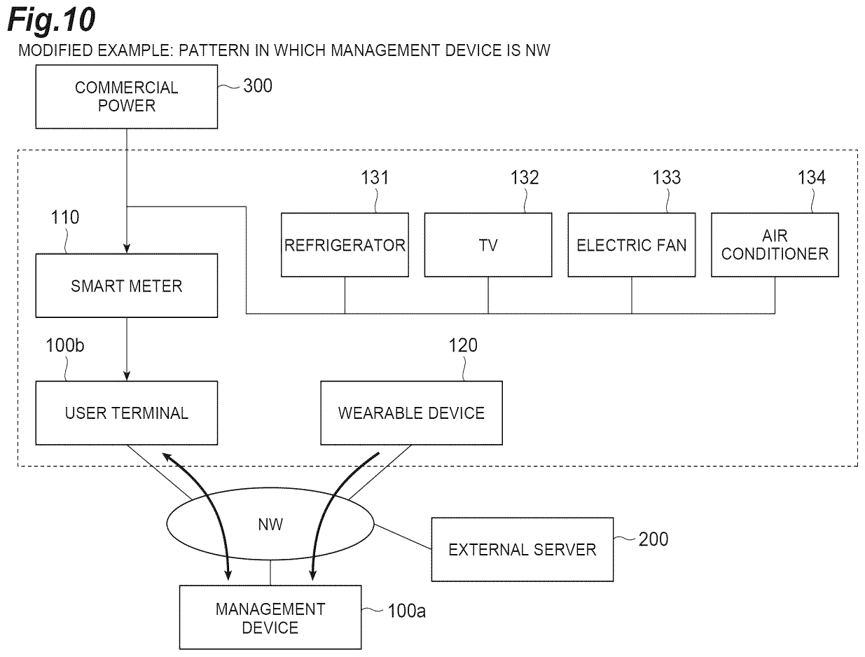

FIG. 10 is a diagram showing a system configuration including a management device 100a in a modified example. As shown in FIG. 19, the wearable device 120 transmits biometric information and the like to the management device 100a via a network. The management device 100a provides a notification indicating the in-use equipment determined on the basis of the biometric information to a user terminal 100b. In the user terminal 100b, the user selects whether or not the in-use equipment notification is correct and transmits a response to the management device 100. The management device 100 updates the log database 104 on the basis of the correct or incorrect in-use equipment notification.

Next, the update of the log database 104 when the entry of the in-use equipment serving as an estimation target has been newly added or discarded in the management device 100 of the present disclosure will be described.

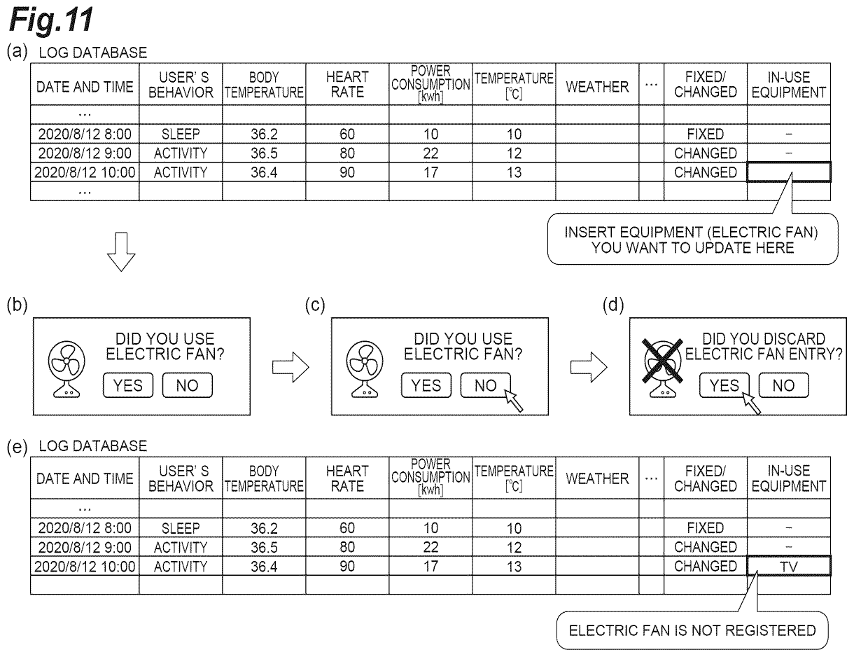

FIG. 11 is a diagram showing a display example of the log database 104 and the display unit 105.

As shown in FIG. 11(a), an attempt is made to estimate the in-use equipment at 10:00 on Aug. 12, 2020, in the log database 104. As shown in FIG. 11(b), the display unit 105 displays an inquiry to confirm the in-use equipment. When the user selects “NO” on the display (FIG. 11(c)), the display unit 105 inquires whether the entry of the in-use equipment has been discarded. When a response for the discarding is positive here (FIG. 11(d)), the log data management unit 108 does not reflect the in-use equipment in the log database 104. Thereafter, the discarded in-use equipment entry is not displayed on the display unit 105.

In FIG. 11(e), a TV is registered as the in-use equipment. The TV is registered as the in-use equipment according to a result of asking the user whether the TV has been used. In addition, because the inquiry is made randomly, the inquiry is not necessarily made about the use of the TV.

Moreover, if discarding has been selected in FIG. 11(d), new in-use equipment that is actually being used may be added and registered in place of the discarded in-use equipment entry. In this case, the added equipment is registered as the in-use equipment in the log database shown in FIG. 11(e).

Next, the operations and effects of the management device 100 (and the management device 100a) in the present disclosure will be described.

The management device 100 or the management device 100a of the present disclosure functions as an in-use equipment estimation device. Unless otherwise specified hereinafter, the management device 100 is assumed to include the management device 100a. In the management device 100, the communication unit 101 acquires biometric information of the user from the wearable device 120 worn by the user. The acquired biometric information is stored in the log database 104.

When an estimation timing is reached, the estimation unit 102 estimates equipment being used by the user on the basis of the biometric information stored in the log database 104.

With this configuration, the in-use equipment at home or the like can be estimated on the basis of biometric information acquired from the wearable device 120 constantly held by the user. Therefore, the in-use equipment can be estimated without using special sensors and the like.

The biometric information used to estimate the in-use equipment includes, for example, at least one of the user's body temperature, heart rate, and blood pressure. These are information items that affect the user's activity and hence affect the in-use equipment. Of course, other biometric information may be included.

In the management device 100 of the present disclosure, the communication unit 101 and the situation determination unit 107 function as a power consumption acquisition unit and acquire the power consumption type (changed/fixed). The estimation unit 102 estimates the in-use equipment on the basis of the power consumption type. This power consumption type is information determined on the basis of whether or not the power consumption changes with the user's intention. For example, the power consumption of a TV is treated as changed power consumption. This is because the TV is used according to the user's intention. On the other hand, the power consumption of a refrigerator is treated as fixed power consumption. This is because the power consumption does not change with the user's intention and is treated as fixed power consumption.

The power consumption acquisition unit acquires the power consumption type, which is one of external information items, on the basis of the user's behavior (whether the user is in the sleeping state), the user's location (whether the user is at a residence), and the power consumption.

With this configuration, the estimation accuracy can be improved by estimating the in-use equipment according to the power consumption type.

In the management device 100 of the present disclosure, the communication unit 101 functions as an external information acquisition unit. The estimation unit 102 estimates the in-use equipment on the basis of the external information. The external information includes at least one of weather, temperature, humidity, and precipitation.

With this configuration, the estimation accuracy of the in-use equipment can be improved by external information such as weather. Weather and the like can affect the in-use equipment, and therefore the accuracy of the estimation can be improved.

The management device 100 of the present disclosure further includes a log database 104 that functions as a log storage unit that stores biometric information in units of predetermined times. The estimation unit 102 estimates the equipment in use on the basis of a change in the biometric information.

Although an actual measurement value indicating biometric information and the like may be used in the present disclosure, the accuracy can be improved by performing an estimation process on the basis of a change from the most recent situation. A change in the biometric information, for example, an increase in the body temperature, an increase in the heart rate, or the like, is affected by the equipment being used by the user. For example, turning on an electric fan will cause the body temperature to decrease. Therefore, the accuracy of the estimation can be improved by observing the change in the biometric information or the like.

In the present disclosure, the log database 104 stores, for example, biometric information, external information, and power consumption type for each hour. The estimation unit 102 calculates changes (an increase, a decrease, a slight increase, a slight decrease, maintenance, and the like) in the biometric information stored in chronological order in the log database 104, and estimates the in-use equipment on the basis of the change, thereby improving the accuracy.

The estimation unit 102 estimates the equipment being used by the user using an estimation model trained with at least the biometric information as an explanatory variable and the in-use equipment as an objective variable. In addition, when the external information and the power consumption type are used as estimation parameters, they are input as explanatory variables and learned.

The management device 100 (100a), which is an in-use equipment estimation device in the present invention, has the following configuration.

[1]

An in-use equipment estimation device comprising:
a biometric information acquisition unit configured to acquire a user's biometric information from a wearable device worn by the user; and
an estimation unit configured to estimate equipment being used by the user on the basis of the biometric information.
   [2]

The in-use equipment estimation device according to [1], wherein the biometric information includes at least one of the user's body temperature, heart rate, and blood pressure.

[3]

The in-use equipment estimation device according to [1] or [2], further comprising an external information acquisition unit configured to acquire external information affecting the equipment being used,
wherein the estimation unit estimates the equipment being used on the basis of the external information in addition to the biometric information.
   [4]

The in-use equipment estimation device according to [3],
wherein the external information acquisition unit acquires a power consumption type (variable, fixed), and
wherein the estimation unit estimates the equipment being used on the basis of the power consumption type.
   [5]

The in-use equipment estimation device according to [4], wherein the power consumption type is determined according to whether or not power consumption changes with the user's intention.

[6]

The in-use equipment estimation device according to any one of [3] to [5], wherein the external information acquisition unit acquires a power consumption type based on the user's behavior, the user's location, and power consumption.

[7]

The in-use equipment estimation device according to any one of [3] to [6], wherein the external information acquisition unit includes at least one of weather, temperature, humidity, and precipitation as the external information.

[8]

The in-use equipment estimation device according to any one of [1] to [7], further comprising a log storage unit configured to store the biometric information in units of predetermined times,
wherein the estimation unit estimates the equipment being used on the basis of a change in the biometric information.
   [9]

The in-use equipment estimation device according to any one of [1] to [8], wherein the estimation unit estimates the equipment being used by the user using an estimation model trained with the biometric information as an explanatory variable and the equipment being used as an objective variable.

The block diagram used for the description of the above embodiments shows blocks of functions. Those functional blocks (component parts) are implemented by any combination of at least one of hardware and software. Further, a means of implementing each functional block is not particularly limited. Specifically, each functional block may be implemented by one physically or logically combined device or may be implemented by two or more physically or logically separated devices that are directly or indirectly connected (e.g., by using wired or wireless connection etc.). The functional blocks may be implemented by combining software with the above-described one device or the above-described plurality of devices.

The functions include determining, deciding, judging, calculating, computing, processing, deriving, investigating, looking up/searching/inquiring, ascertaining, receiving, transmitting, outputting, accessing, resolving, selecting, choosing, establishing, comparing, assuming, expecting, considering, broadcasting, notifying, communicating, forwarding, configuring, reconfiguring, allocating/mapping, assigning and the like, though not limited thereto. For example, the functional block (component part) that implements the function of transmitting is referred to as a transmitting unit or a transmitter. In any case, a means of implementation is not particularly limited as described above.

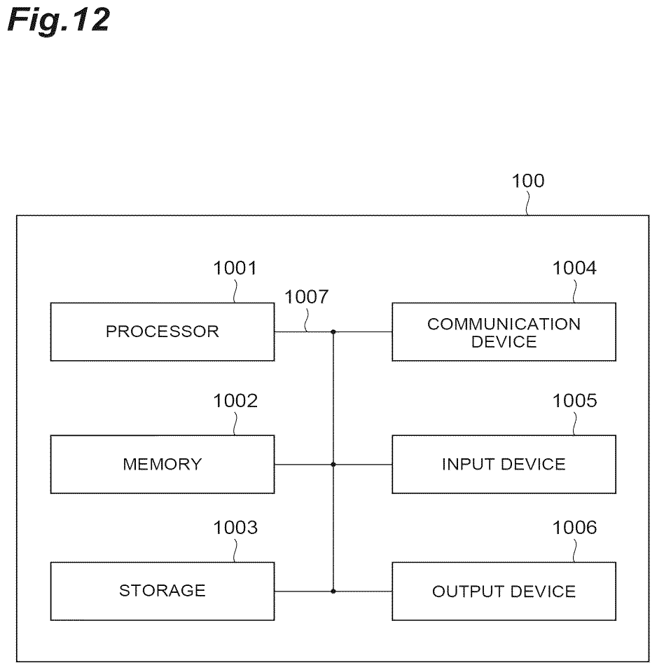

For example, the management device 100 and the management device 100a and the like (hereinafter referred to as the management device 100) according to one embodiment of the present disclosure may function as a computer that performs processing of an in-use equipment estimation method according to the present disclosure. FIG. 12 is a view showing an example of the hardware configuration of the management device 100 according to one embodiment of the present disclosure. The management device 100 described above may be physically configured as a computer device that includes a processor 1001, a memory 1002, a storage 1003, a communication device 1004, an input device 1005, an output device 1006, a bus 1007 and the like.

In the following description, the term “device” may be replaced with a circuit, a device, a unit, or the like. The hardware configuration of the management device 100 may be configured to include one or a plurality of the devices shown in the drawings or may be configured without including some of those devices.

The functions of the management device 100 may be implemented by loading predetermined software (programs) on hardware such as the processor 1001 and the memory 1002, so that the processor 1001 performs computations to control communications by the communication device 1004 and control at least one of reading and writing of data in the memory 1002 and the storage 1003.

The processor 1001 may, for example, operate an operating system to control the entire computer. The processor 1001 may be configured to include a CPU (Central Processing Unit) including an interface with a peripheral device, a control device, an arithmetic device, a register and the like. For example, the estimation unit 102, the learning unit 106, the situation determination unit 107, and a log data management unit 108 and the like described above may be implemented by the processor 1001.

Further, the processor 1001 loads a program (program code), a software module and data from at least one of the storage 1003 and the communication device 1004 into the memory 1002 and performs various processing according to them. As the program, a program that causes a computer to execute at least some of the operations described in the above embodiments is used. For example, the estimation unit 102, the learning unit 106, the situation determination unit 107, and a log data management unit 108 may be implemented by a control program that is stored in the memory 1002 and operates on the processor 1001, and the other functional blocks may be implemented in the same way. Although the above-described processing is executed by one processor 1001 in the above description, the processing may be executed simultaneously or sequentially by two or more processors 1001. The processor 1001 may be implemented in one or more chips. Note that the program may be transmitted from a network through a telecommunications line.

The memory 1002 is a computer-readable recording medium, and it may be composed of at least one of ROM (Read Only Memory), EPROM (Erasable Programmable ROM), EEPROM (Electrically Erasable Programmable ROM), RAM (Random Access Memory) and the like, for example. The memory 1002 may be also called a register, a cache, a main memory (main storage device) or the like. The memory 1002 can store a program (program code), a software module and the like that can be executed for implementing the in-use equipment estimation method according to one embodiment of the present disclosure.

The storage 1003 is a computer-readable recording medium, and it may be composed of at least one of an optical disk such as a CD-ROM (Compact Disk ROM), a hard disk drive, a flexible disk, a magneto-optical disk (e.g., a compact disk, a digital versatile disk, and a Blu-ray (registered trademark) disk), a smart card, a flash memory (e.g., a card, a stick, and a key drive), a floppy (registered trademark) disk, a magnetic strip and the like, for example. The storage 1003 may be called an auxiliary storage device. The above-described storage medium may be a database, a server, or another appropriate medium including at least one of the memory 1002 and/or the storage 1003, for example.

The communication device 1004 is hardware (a transmitting and receiving device) for performing communication between computers via at least one of a wired network and a wireless network, and it may also be referred to as a network device, a network controller, a network card, a communication module, or the like. The communication device 1004 may include a high-frequency switch, a duplexer, a filter, a frequency synthesizer or the like in order to implement at least one of FDD (Frequency Division Duplex) and TDD (Time Division Duplex), for example. For example, the above-described communication unit 101 may be implemented by the communication device 1004. The communication unit 101 may be implemented in such a way that a transmitting unit and a receiving unit are physically or logically separated.

The input device 1005 is an input device (e.g., a keyboard, a mouse, a microphone, a switch, a button, a sensor, etc.) that receives an input from the outside. The output device 1006 is an output device (e.g., a display, a speaker, an LED lamp, etc.) that makes output to the outside. Note that the input device 1005 and the output device 1006 may be integrated (e.g., a touch panel).

In addition, the devices such as the processor 1001 and the memory 1002 are connected by the bus 1007 for communicating information. The bus 1007 may be a single bus or may be composed of different buses between different devices.

Further, the management device 100 may include hardware such as a microprocessor, a DSP (Digital Signal Processor), an ASIC (Application Specific Integrated Circuit), a PLD (Programmable Logic Device), and an FPGA (Field Programmable Gate Array), and some or all of the functional blocks may be implemented by the above-described hardware components. For example, the processor 1001 may be implemented with at least one of these hardware components.

Notification of information may be made by another method, not limited to the aspects/embodiments described in the present disclosure. For example, notification of information may be made by physical layer signaling (e.g., DCI (Downlink Control Information), UCI (Uplink Control Information)), upper layer signaling (e.g., RRC (Radio Resource Control) signaling, MAC (Medium Access Control) signaling, annunciation information (MIB (Master Information Block), SIB (System Information Block))), another signal, or a combination of them. Further, RRC signaling may be called an RRC message, and it may be an RRC Connection Setup message, an RRC Connection Reconfiguration message or the like, for example.

The procedure, the sequence, the flowchart and the like in each of the aspects/embodiments described in the present disclosure may be in a different order unless inconsistency arises. For example, for the method described in the present disclosure, elements of various steps are described in an exemplified order, and it is not limited to the specific order described above.

Input/output information or the like may be stored in a specific location (e.g., memory) or managed in a management table. Further, input/output information or the like can be overwritten or updated, or additional data can be written. Output information or the like may be deleted. Input information or the like may be transmitted to another device.

The determination may be made by a value represented by one bit (0 or 1), by a truth-value (Boolean: true or false), or by numerical comparison (e.g., comparison with a specified value).

Each of the aspects/embodiments described in the present disclosure may be used alone, may be used in combination, or may be used by being switched according to the execution. Further, a notification of specified information (e.g., a notification of “being X”) is not limited to be made explicitly, and it may be made implicitly (e.g., a notification of the specified information is not made).

Although the present disclosure is described in detail above, it is apparent to those skilled in the art that the present disclosure is not restricted to the embodiments described in this disclosure. The present disclosure can be implemented as a modified and changed form without deviating from the spirit and scope of the present disclosure defined by the appended claims. Accordingly, the description of the present disclosure is given merely by way of illustration and does not have any restrictive meaning to the present disclosure.

Software may be called any of software, firmware, middleware, microcode, hardware description language or another name, and it should be interpreted widely so as to mean an instruction, an instruction set, a code, a code segment, a program code, a program, a sub-program, a software module, an application, a software application, a software package, a routine, a sub-routine, an object, an executable file, a thread of execution, a procedure, a function and the like.

Further, software, instructions and the like may be transmitted and received via a transmission medium. For example, when software is transmitted from a website, a server or another remote source using at least one of wired technology (a coaxial cable, an optical fiber cable, a twisted pair and a digital subscriber line (DSL) etc.) and wireless technology (infrared rays, microwave etc.), at least one of those wired technology and wireless technology are included in the definition of the transmission medium.

The information, signals and the like described in the present disclosure may be represented by any of various different technologies. For example, data, an instruction, a command, information, a signal, a bit, a symbol, a chip and the like that can be referred to in the above description may be represented by a voltage, a current, an electromagnetic wave, a magnetic field or a magnetic particle, an optical field or a photon, or an arbitrary combination of them.

Note that the term described in the present disclosure and the term needed to understand the present disclosure may be replaced by a term having the same or similar meaning. For example, at least one of a channel and a symbol may be a signal (signaling). Further, a signal may be a message. Furthermore, a component carrier (CC) may be called a cell, a frequency carrier, or the like.

Further, information, parameters and the like described in the present disclosure may be represented by an absolute value, a relative value to a specified value, or corresponding different information. For example, radio resources may be indicated by an index.

The names used for the above-described parameters are not definitive in any way. Further, mathematical expressions and the like using those parameters are different from those explicitly disclosed in the present disclosure in some cases. Because various channels (e.g., PUCCH, PDCCH etc.) and information elements (e.g., TPC etc.) can be identified by every appropriate names, various names assigned to such various channels and information elements are not definitive in any way.

In the present disclosure, the terms such as “Mobile Station (MS)” “user terminal”, “User Equipment (UE)” and “terminal” can be used to be compatible with each other.

The mobile station can be also called, by those skilled in the art, a subscriber station, a mobile unit, a subscriber unit, a wireless unit, a remote unit, a mobile device, a wireless device, a wireless communication device, a remote device, a mobile subscriber station, an access terminal, a mobile terminal, a wireless terminal, a remote terminal, a handset, a user agent, a mobile client, a client or several other appropriate terms.

Note that the term “determining” and “determining” used in the present disclosure includes a variety of operations. For example, “determining” and “determining” can include regarding the act of judging, calculating, computing, processing, deriving, investigating, looking up/searching/inquiring (e.g., looking up in a table, a database or another data structure), ascertaining or the like as being “determined” and “determined”. Further, “determining” and “determining” can include regarding the act of receiving (e.g., receiving information), transmitting (e.g., transmitting information), inputting, outputting, accessing (e.g., accessing data in a memory) or the like as being “determined” and “determined”. Further, “determining” and “determining” can include regarding the act of resolving, selecting, choosing, establishing, comparing or the like as being “determined” and “determined”. In other words, “determining” and “determining” can include regarding a certain operation as being “determined” and “determined”. Further, “determining (determining)” may be replaced with “assuming”, “expecting”, “considering” and the like.

The term “connected”, “coupled” or every transformation of this term means every direct or indirect connection or coupling between two or more elements, and it includes the case where there are one or more intermediate elements between two elements that are “connected” or “coupled” to each other. The coupling or connection between elements may be physical, logical, or a combination of them. For example, “connect” may be replaced with “access”. When used in the present disclosure, it is considered that two elements are “connected” or “coupled” to each other by using at least one of one or more electric wires, cables, and printed electric connections and, as several non-definitive and non-comprehensive examples, by using electromagnetic energy such as electromagnetic energy having a wavelength of a radio frequency region, a microwave region and an optical (both visible and invisible) region.

The description “on the basis of” used in the present disclosure does not mean “only on the basis of” unless otherwise noted. In other words, the description “on the basis of” means both of “only on the basis of” and “at least on the basis of”.

When the terms such as “first” and “second” are used in the present disclosure, any reference to the element does not limit the amount or order of the elements in general. Those terms can be used in the present disclosure as a convenient way to distinguish between two or more elements. Thus, reference to the first and second elements does not mean that only two elements can be adopted or the first element needs to precede the second element in a certain form.

As long as “include”, “including” and transformation of them are used in the present disclosure, those terms are intended to be comprehensive like the term “comprising”. Further, the term “or” used in the present disclosure is intended not to be exclusive OR.

In the present disclosure, when articles, such as “a”, “an”, and “the” in English, for example, are added by translation, the present disclosure may include that nouns following such articles are plural.

In the present disclosure, the term “A and B are different” may mean that “A and B are different from each other”. Note that this term may mean that “A and B are different from C”. The terms such as “separated” and “coupled” may be also interpreted in the same manner.

### REFERENCE SIGNS LIST

100 Management device
120 Wearable device
200 External server
131 Refrigerator
132 TV
133 Electric fan
134 Air conditioner
300 Commercial power
110 Smart meter
101 Communication unit
102 Estimation unit
103 Estimation model
104 Log database
105 Display unit
106 Learning unit
107 Situation determination unit
108 Log data management unit.

1. An in-use equipment estimation device comprising:
a biometric information acquisition unit configured to acquire a user's biometric information from a wearable device worn by the user; and
an estimation unit configured to estimate equipment being used by the user on the basis of the biometric information.

2. The in-use equipment estimation device according to claim 1, wherein the biometric information includes at least one of the user's body temperature, heart rate, and blood pressure.

3. The in-use equipment estimation device according to claim 1, further comprising an external information acquisition unit configured to acquire external information affecting the equipment being used,
wherein the estimation unit estimates the equipment being used on the basis of the external information in addition to the biometric information.

4. The in-use equipment estimation device according to claim 3,
wherein the external information acquisition unit acquires a power consumption type, and
wherein the estimation unit estimates the equipment being used on the basis of the power consumption type.

5. The in-use equipment estimation device according to claim 4, wherein the power consumption type is determined according to whether or not power consumption changes with the user's intention.

6. The in-use equipment estimation device according to claim 3, wherein the external information acquisition unit acquires a power consumption type based on the user's behavior, the user's location, and power consumption.

7. The in-use equipment estimation device according to claim 3, wherein the external information acquisition unit includes at least one of weather, temperature, humidity, and precipitation as the external information.

8. The in-use equipment estimation device according to claim 1, further comprising a log storage unit configured to store the biometric information in units of predetermined times,
wherein the estimation unit estimates the equipment being used on the basis of a change in the biometric information.

9. The in-use equipment estimation device according to claim 1, wherein the estimation unit estimates the equipment being used by the user using an estimation model trained with the biometric information as an explanatory variable and the equipment being used as an objective variable.

10. The in-use equipment estimation device according to claim 9, further comprising a learning unit configured to learn with the biometric information as an explanatory variable and the equipment being used as an objective variable.
